(19) United States
(12) Patent Application Publication
(10) Pub. No.: US 2009/0275908 A1
(43) Pub. Date: Nov. 5, 2009

(54) Absorbent Articles Capable of Indicating the Presence of Urine

(75) Inventor: Xuedong Song, Roswell, GA (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 12/112,301

(22) Filed: Apr. 30, 2008

(51) Int. Cl. A61F 13/42

(52) U.S. Cl. 604/361

(57) ABSTRACT

In accordance with one embodiment of the present disclosure a method for detecting urine is described. The method includes contacting urine with a substrate. The substrate includes charged cellulosic fibers having a color-changing composition immobilized thereon, the color-changing composition including a pH indicator, pH adjuster, and a wettability agent, wherein the pH indicator is configured to change color when contacted with urine. The presence of urine is determined based on a change in color of the pH indicator.

### BACKGROUND

Disposable absorbent articles such as diapers, training pants, incontinence pads, and the like are highly absorbent and efficiently pull moisture away from the wearer, reducing skin irritation caused by prolonged wetness exposure. However, because these articles are so absorbent, wearers may not realize they have urinated, particularly if they are inexperienced toddlers who may not recognize the meaning of body sensations associated with urination. Thus, the wearer may not recognize their urination control failure or be aware the article should be changed. Furthermore, parents or caregivers may not recognize that the absorbent article requires changing.

Visual mechanisms have been employed to signal the presence of wetness in absorbent articles. There are a large number of wetness sensing technologies that currently exist which utilize such visual mechanisms. For instance, recent wetness sensing technology employs highly charged porous nylon membranes to immobilize pH indicators that can change color in the presence of urine. The color change contrast in highly charged porous nylon membranes is greater than that found in conventional technologies. However, despite their excellent sensing performance, nylon membranes can be very costly.

Materials formed from cellulose fibers are more readily available and are relatively inexpensive when compared to nylon membranes. Cellulose fibers can be modified to have high density surface charges and can also be used to make porous webs.

Thus, a need exists for cellulose fiber-based materials that can effectively utilize similar color change mechanisms to that found in nylon membranes to indicate the presence of urine. An absorbent article that incorporates such materials would be particularly beneficial.

### SUMMARY

In accordance with one embodiment of the present disclosure a method for detecting urine is described. The method includes contacting urine with a substrate. The substrate includes charged cellulosic fibers having a color-changing composition immobilized thereon, the color-changing composition including a pH indicator, a pH adjuster, and a wettability agent, wherein the pH indicator is configured to change color when contacted with urine. The presence of urine is determined based on a change in color of the pH indicator.

In still another embodiment of the present disclosure, an absorbent article capable of determining the presence of urine is described. The absorbent article includes a substantially liquid impermeable layer, a liquid permeable layer, an absorbent core positioned between the substantially liquid impermeable layer and the liquid permeable layer, and a charged cellulosic fibers integrated into the article and positioned such that the charged cellulosic fibers are in fluid communication with the urine when provided by a wearer of the article. The charged cellulosic fibers have a color-changing composition immobilized thereon, the color-changing composition including a pH indicator, a pH adjuster, and a wettability agent, wherein the indicator is configured to change color when contacted with urine.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended FIGURE in which:

FIG. 1 is a perspective view of one embodiment of an absorbent article that can be used in the present disclosure.

Repeat use of reference characters in the present specification and drawings is intended to represent same or analogous features or elements of the disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to materials formed from cellulose fiber that are capable of indicating the presence of urine. Immobilized pH indicators are utilized with the materials to accurately indicate the presence of urine. It has been determined that the poor performance of conventional cellulose fiber-based substrates results from low indicator loading capacity and poor urine wettability. Certain conventional materials do not immobilize pH indicators resulting in leaching a diffusion of such indicators. The present disclosure describes substrates formed from cellulose fibers that are treated with charged polymers, the substrates having improved indicator loading capacity. In addition, the materials of the present disclosure include a color-changing composition having a pH indicator, a pH adjuster, and a wettability agent that provides improved urine wettability. The materials described herein can be made for low cost, while still exhibiting very good performance.

The present disclosure provides a simple, user-friendly, cost-effective approach for rapid determination of the presence of urine. Additionally, the materials described herein can be incorporated into absorbent articles such as diapers and incontinent pads to assist in determining urine presence.

Generally speaking, the present disclosure describes a substrate that includes charged cellulosic fibers coated with a color-changing composition to indicate the presence of urine. The substrate can be formed in a manner so that urine is capable of passing therethrough.

The substrate can be a porous web formed from naturally occurring materials, such as polysaccharides (e.g., cellulose materials such as paper and cellulose derivatives, such as cellulose acetate and nitrocellulose). For example, the substrate can include fibers formed by a variety of pulping processes, such as kraft pulp, sulfite pulp, thermomechanical pulp, etc. The pulp fibers may include softwood fibers having an average fiber length of greater than 1 mm and particularly from about 2 to 5 mm based on a length-weighted average. Such softwood fibers can include, but are not limited to, northern softwood, southern softwood, redwood, red cedar, hemlock, pine (e.g., southern pines), spruce (e.g., black spruce), combinations thereof, and the like. Exemplary commercially available pulp fibers suitable for the present invention include those available from Kimberly-Clark Corporation under the trade designations “Longlac-19”.

Hardwood fibers, such as eucalyptus, maple, birch, aspen, and the like, can also be used. In certain instances, eucalyptus fibers may be particularly desired because the fibers are thin and not highly mobilized. Moreover, if desired, secondary fibers obtained from recycled materials may be used, such as fiber pulp from sources such as, for example, newsprint, reclaimed paperboard, and office waste. Further, other natural fibers can also be used in the present invention, such as abaca, sabai grass, milkweed floss, pineapple leaf, and the like.

In addition, in some instances, synthetic fibers can also be utilized in combination with the cellulosic fibers described herein. Some suitable synthetic fibers can include, but are not limited to, rayon fibers, ethylene vinyl alcohol copolymer fibers, polyolefin fibers, polyesters, and the like. As used herein, “synthetic fibers” refer to man-made, polymeric fibers that may comprise one or more polymers, each of which may have been generated from one or more monomers. The polymeric materials in the synthetic fibers may independently be thermoplastic, thermosetting, elastomeric, non-elastomeric, crimped, substantially uncrimped, colored, uncolored, filled with filler materials or unfilled, birefringent, circular in cross-section, multilobal or otherwise non-circular in cross-section, and so forth. Synthetic fibers can be produced by any known technique. Synthetic fibers can be monocomponent fibers such as filaments of polyesters, polyolefins or other thermoplastic materials, or may be bicomponent or multicomponent fibers. When more than one polymer is present in a fiber, the polymers may be blended, segregated in microscopic or macroscopic phases, present in side-by-side or sheath-core structures, or distributed in any way known in the art.

It should be understood that any suitable method of forming a web is contemplated for use in the present disclosure. The size and shape of the substrate can generally vary as is readily recognized by those skilled in the art and also depending on the method of forming the web utilized. For instance, a porous web can have a length of from about 10 to about 100 millimeters, in some embodiments from about 20 to about 80 millimeters, and in some embodiments, from about 40 to about 60 millimeters. The width of the web can also range from about 0.5 to about 20 millimeters, in some embodiments from about 1 to about 15 millimeters, and in some embodiments, from about 2 to about 10 millimeters. The thickness of the web can be less than about 500 micrometers, in some embodiments less than about 250 micrometers, and in some embodiments, less than about 150 micrometers. The size and shape of the substrate should be sufficient so that the color change occurring thereon is visible.

As discussed above, the fibers of the present disclosure can be treated with a charged polymer prior to contact with a color-changing composition. For instance, it has been found that wet and dry strength agents can be utilized advantageously in connection with the present disclosure. As used herein, “wet strength agents” refer to materials used to immobilize the bonds between fibers in the wet state. Any material that when added to a web results in providing the tissue sheet with a mean wet geometric tensile strength:dry geometric tensile strength ratio in excess of about 0.1 is, for purposes of the present disclosure, termed a wet strength agent. Typically these materials are referred to as permanent wet strength agents or as “temporary” wet strength agents. For the purposes of differentiating permanent wet strength agents from temporary wet strength agents, the permanent wet strength agents will be defined as those resins which, when incorporated into a web, will provide a web that retains more than 50 percent of its original wet strength after exposure to water for a period of at least five minutes. Temporary wet strength agents are those which show about 50 percent or less of their original wet strength after being saturated with water for five minutes. Both classes of wet strength agents may find application for the materials of the present disclosure. If present, the amount of wet strength agent added to the pulp fibers can be about 0.1 dry weight percent or greater, more specifically about 0.2 dry weight percent or greater, and still more specifically from about 0.1 to about 3 dry weight percent, based on the dry weight of the fibers. Suggested methods of treatment include, but are not limited to, saturation, spray, slot die, printing, foaming, and combinations and modifications thereof.

The temporary wet strength agents may be cationic or anionic. Such compounds include, without limitation, PAREZ™ 631 NC and PAREZ® 725 temporary wet strength resins that are cationic glyoxylated polyacrylamide available from Cytec Industries (West Paterson, N.J.). Hercobond 1366, manufactured by Hercules, Inc., located at Wilmington, Del., is another commercially available cationic glyoxylated polyacrylamide that may be used in accordance with the present disclosure. Additional examples of temporary wet strength agents include dialdehyde starches such as Cobond® 1000 from National Starch and Chemical Company and other aldehyde containing polymers known in the art.

Suitable permanent wet strength agents include cationic oligomeric or polymeric resins. Polyamide-polyamine-epichlorohydrin type resins, such as KYMENE 557H sold by Hercules, Inc., located at Wilmington, Del., or other resins sold under the KYMENE designation, are widely used permanent wet-strength agents. Other cationic resins include polyethylenimine resins and aminoplast resins obtained by reaction of formaldehyde with melamine or urea.

Once treated with a charged polymer, a color-changing composition of the present disclosure can be applied to the fibers using any known application technique. Desirably, the color-changing composition is applied to the fibers before the fibers are incorporated into a web or combined with other fibers into a web. Again, suggested methods of treatment include, but are not limited to, saturation, spray, slot die, printing, foaming, and combinations and modifications thereof.

The color-changing composition of the present disclosure includes a pH indicator. Various solvents can be utilized to form a solution with the pH indicator, such as, but not limited to, water, acetonitrile, dimethylsulfoxide (DMSO), ethyl alcohol, dimethylformamide (DMF), and other polar organic solvents. The amount of the pH indicator in the solution can range from about 0.001 to about 100 milligrams per milliliter of solvent, and in some embodiments, from about 0.1 to about 10 milligrams per milliliter of solvent. The pH indicator concentration can be selectively controlled to provide the desired level of detection sensitivity.

In certain embodiments of the present disclosure, a crosslinked network containing the pH indicator is formed on a substrate. Without intending to be limited by theory, it is believed that the crosslinked network can help durably secure the pH indicator, thereby allowing a user to more readily detect a change in its color during use. The crosslinked network can contain “intra-cross links” (i.e., covalent bonds between functional groups of a single molecule) and/or “inter-cross links” (i.e., covalent bonds between different molecules, e.g., between two pH indicator molecules or between a pH indicator molecule and the substrate surface). Crosslinking can be carried out via self crosslinking of the indicator and/or through the inclusion of a separate crosslinking agent. Suitable crosslinking agents, for instance, can include polyglycidyl ethers, such as ethylene glycol diglycidyl ether and polyethylene glycol diglycidyl ether; acrylamides; compounds containing one or more hydrolyzable groups, such as alkoxy groups (e.g., methoxy, ethoxy and propoxy); alkoxyalkoxy groups (e.g., methoxyethoxy, ethoxyethoxy and methoxypropoxy); acyloxy groups (e.g., acetoxy and octanoyloxy); ketoxime groups (e.g., dimethylketoxime, methylketoxime and methylethylketoxime); alkenyloxy groups (e.g., vinyloxy, isopropenyloxy, and 1-ethyl-2-methylvinyloxy); amino groups (e.g., dimethylamino, diethylamino and butylamino); aminoxy groups (e.g., dimethylaminoxy and diethylaminoxy); and amide groups (e.g., N-methylacetamide and N-ethylacetamide).

Any of a variety of different crosslinking mechanisms can be employed in the present disclosure, such as thermal initiation (e.g., condensation reactions, addition reactions, etc.), electromagnetic radiation, and so forth. Some suitable examples of electromagnetic radiation that can be used in the present disclosure include, but are not limited to, electron beam radiation, natural and artificial radio isotopes (e.g., α, β, and γ rays), x-rays, neutron beams, positively-charged beams, laser beams, ultraviolet, etc. Electron beam radiation, for instance, involves the production of accelerated electrons by an electron beam device. Electron beam devices are generally well known in the art. For instance, in one embodiment, an electron beam device can be used that is available from Energy Sciences, Inc., of Woburn, Mass. under the name “Microbeam LV.” Other examples of suitable electron beam devices are described in U.S. Pat. No. 5,003,178 to Livesay; U.S. Pat. No. 5,962,995 to Avnerv; U.S. Pat. No. 6,407,492 to Avnerv, et al., which are incorporated herein in their entirety by reference thereto for all purposes. The wavelength λ of the radiation can vary for different types of radiation of the electromagnetic radiation spectrum, such as from about 10⁻¹⁴ meters to about 10⁻⁵ meters. Electron beam radiation, for instance, has a wavelength λ of from about 10⁻¹³ meters to about 10⁻⁹ meters. Besides selecting the particular wavelength λ of the electromagnetic radiation, other parameters can also be selected to control the degree of crosslinking. For example, the dosage can range from about 0.1 megarads (Mrads) to about 10 Mrads, and in some embodiments, from about 1 Mrads to about 5 Mrads.

The source of electromagnetic radiation can be any radiation source known to those of ordinary skill in the art. For example, an excimer lamp or a mercury lamp with a D-bulb can be used. Other specialty-doped lamps that emit radiation at a fairly narrow emission peak can be used with photoinitiators which have an equivalent absorption maximum. For example, the V-bulb, available from Fusion Systems, is another suitable lamp for use. In addition, specialty lamps having a specific emission band can be manufactured for use with one or more specific photoinitiators.

Initiators can be employed in some embodiments that enhance the functionality of the selected crosslinking technique. Thermal initiators, for instance, can be employed in certain embodiments, such as azo, peroxide, persulfate, and redox initiators. Representative examples of suitable thermal initiators include azo initiators such as 2,2′-azobis(2,4-dimethylvaleronitrile), 2,2′-azobis(isobutyronitrile), 2,2′-azobis-2-methylbutyronitrile, 1,1′-azobis(1-cyclohexanecarbonitrile), 2,2′-azobis(methyl isobutyrate), 2,2′-azobis(2-amidinopropane) dihydrochloride, and 2,2′-azobis(4-methoxy-2,4-dimethylvaleronitrile); peroxide initiators such as benzoyl peroxide, acetyl peroxide, lauroyl peroxide, decanoyl peroxide, dicetyl peroxydicarbonate, di(4-t-butylcyclohexyl) peroxydicarbonate, di(2-ethylhexyl) peroxydicarbonate, t-butylperoxypivalate, t-butylperoxy-2-ethylhexanoate, and dicumyl peroxide; persulfate initiators such as potassium persulfate, sodium persulfate, and ammonium persulfate; redox (oxidation-reduction) initiators such as combinations of the above persulfate initiators with reducing agents such as sodium metabisulfite and sodium bisulfite, systems based on organic peroxides and tertiary amines, and systems based on organic hydroperoxides and transition metals; other initiators such as pinacols; and the like (and mixtures thereof). Azo compounds and peroxides are generally preferred. Photoinitiators can likewise be employed, such as substituted acetophenones, such as benzyl dimethyl ketal and 1-hydroxycyclohexyl phenyl ketone; substituted alpha-ketols, such as 2-methyl-2-hydroxypropiophenone; benzoin ethers, such as benzoin methyl ether and benzoin isopropyl ether; substituted benzoin ethers, such as anisoin methyl ether; aromatic sulfonyl chlorides; photoactive oximes; and so forth (and mixtures thereof. Other suitable photoinitiators can be described in U.S. Pat. No. 6,486,227 to Nohr, et al. and U.S. Pat. No. 6,780,896 to MacDonald, et al., both of which are incorporated herein by reference.

Although not required, additional components can also be employed within the crosslinked network to facilitate the securement of the pH indicator. For example, an anchoring compound can be employed that links the pH indicator to the surface of the substrate and further improves the durability of the pH indicator on the substrate. Typically, the anchoring compound is larger in size than the pH indicator, which improves its likelihood of remaining on the surface of the substrate during use. For example, the anchoring compound can include a macromolecular compound, such as a polymer, oligomer, dendrimer, particle, etc. Polymeric anchoring compounds can be natural, synthetic, or combinations thereof.

The manner in which the anchoring compound is used to link the pH indicator and the substrate can vary. In one embodiment, for instance, the anchoring compound is attached to the pH indicator as part of the color-changing composition, prior to application of the color-changing composition to the cellulosic fibers. In other embodiments, the anchoring compound can be bonded to the cellulosic fibers prior to application of the color-changing composition. In still other embodiments, the materials can be applied as separate components to the substrate and attachment reactions can take place in situ, optionally at the same time as the crosslinking of the network. For instance, the pH indicator can bind the anchoring compound, the anchoring compound can bind the substrate, and simultaneously, cross-linking reactions can take place between anchoring compounds, between indicators, or between the two. In one such embodiment, the cross-linked network thus formed can be physically held on a porous membrane of the substrate without the need for bonding between the porous membrane and the other components of the system. In particular, the crosslinked network, portions of which can extend within and among the pores of the porous membrane, can be physically constrained on the substrate, even without specific bonds forming between the substrate and the components of the crosslinked network.

It should be understood that, besides covalent bonding, other attachment techniques, such as charge-charge interactions, can also be utilized for attaching the anchoring compound to the substrate and/or for attaching the pH indicator of the color-changing composition to the anchoring compound. For instance, a charged anchoring compound, such as a positively charged anchoring compound, can be immobilized on a negatively charged substrate, through charge-charge interactions between the two. Similarly, a negatively charged indicator can be immobilized on a positively charged anchoring compound.

It is important to select a pH indicator that has sensitivity towards the pH change caused by the urine. Since normal urine pH lies from about 5.5 to about 10.5, useful pH indicators can have a significant color transition either at less than about 5.5 or greater than about 10.5. The pH indicator color transition pH is preferred to lie from about 1.0 to about 5.5 or from about 10.5 to about 11.5.

For instance, in certain embodiments, phthalein chromogens constitute one class of suitable pH-sensitive chromogens that can be employed in the present disclosure. Exemplary pH indicators include Bromophenol Blue (3′,3″,5′,5″-tetrabromophenolsulfonephthalein), Bromochlorophenol Blue (the sodium salt of dibromo-5′,5″-dichlorophenolsulfonephthalein), Bromocresol Green (3′,3″,5′,5″-tetrabromo-ortho-cresolsulfonephthalein), and so forth. Still other suitable phthalein chromogens are well known in the art, and can include thymolphthalein. Thymolphthalein exhibits a transition from colorless to blue over a pH range of about 9.4 to 10.6; Bromophenol Blue exhibits a transition from yellow to violet over a pH range of about 3.0 to 4.6; Bromocresol Green exhibits a transition from yellow to blue over a pH range of about 3.8 to 5.4.

Anthraquinones constitute another suitable class of pH-sensitive chromogens for use in the present disclosure. Anthraquinones have the following general structure:

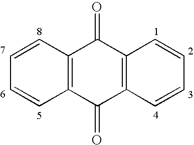

The numbers 1-8 shown in the general formula represent a location on the fused ring structure at which substitution of a functional group can occur. Some examples of such functional groups that can be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of quinones can be formed with varying colors and intensities. Other functional groups, such as sulfonic acids, can also be used to render certain types of compounds (e.g., higher molecular weight anthraquinones) water-soluble.

Some suitable anthraquinones that can be used in the present disclosure include, Mordant, Alizarin, and so forth. For instance, Alizarin Yellow R, exhibits a transition from yellow to orange-red over a pH range of about 10.1 to 12.0.

Yet another suitable class of pH-sensitive chromogens that can be employed is aromatic azo compounds having the general structure:

X—R₁—N═N—R₂—Y

wherein,
R₁ is an aromatic group;
R₂ is selected from the group consisting of aliphatic and aromatic groups; and
X and Y are independently selected from the group consisting of hydrogen, halides, —NO₂, —NH₂, aryl groups, alkyl groups, alkoxy groups, sulfonate groups, —SO₃H, —OH, —COH, —COOH, halides, etc. Also suitable are azo derivatives, such as azoxy compounds (X—R₁—N═NO—R₂—Y) or hydrazo compounds (X—R₁—NH—NH—R₂—Y). Particular examples of such azo compounds (or derivatives thereof) include Methyl Violet 2B, Methyl Yellow, Methyl Orange, Methyl Red, and Methyl Green. For instance, Methyl Violet 2B undergoes a transition from yellow to blue-violet at a pH range of about 0 to 1.6, Methyl Yellow undergoes a transition from red to yellow at a pH range of about 2.9 to 4.0, Methyl Orange undergoes a transition from red to yellow at a pH range of about 3.1 to 4.4.

Arylmethanes (e.g., diarylmethanes and triarylmethanes) constitute still another class of suitable pH-sensitive chromogens for use in the present disclosure. For example, Malachite Green typically exhibits a transition from yellow to blue-green over a pH range 0.2 to 1.8. Above a pH of about 1.8, malachite green turns a deep green color.

Still other suitable pH-sensitive chromogens that can be employed include Congo Red, Methylene Blue, Acid Fuchsin, Indigo Carmine, Picric acid, Metanil Yellow, Phloxine B, 2,4-dinitrophenol, Nile Blue A, and so forth. For instance, Congo Red undergoes a transition from blue to red at a pH range of about 3.0 to 5.2, and Neutral Red undergoes a transition from red to yellow at a pH range of about 11.4 to 13.0.

However, any suitable pH indicator as would be known in the art is contemplated for use in the present disclosure.

In certain embodiments, the initial color of the immobilized indicator can be easily adjusted by immobilizing the indicator along with a pH adjuster in the color-changing composition either an acid, a buffer, a base or some combination thereof. The initial color is important to provide a sharp color contrast as large as possible. For instance, when bromophenol blue is used as an indicator, an acid condition gives the indicator zone a yellow color, which is clearly distinguishable from blue color under the pH range of about 5.5 to about 10.5 for normal urine.

Suitable pH adjusters can include any reagent that can provide a pH for the composition ranging from about pH of less than about 5.5 or greater than about 10.5. Additionally, other suitable pH adjusters can include sulfonic acids (e.g., 2-[N-morpholino]ethane sulfonic acid (“MES”), carboxylic acids, and polymeric acids. Specific examples of suitable carboxylic acids are citric acid, glycolic acid, lactic acid, acetic acid, maleic acid, gallic acid, malic acid, succinic acid, glutaric acid, benzoic acid, malonic acid, salicylic acid, gluconic acid, and mixtures thereof. Specific examples of suitable polymeric acids include straight-chain poly(acrylic) acid and its copolymers (e.g., maleic-acrylic, sulfonic-acrylic, and styrene-acrylic copolymers), cross-linked polyacrylic acids having a molecular weight of less than about 250,000, poly(methacrylic) acid, and naturally occurring polymeric acids such as carageenic acid, carboxymethyl cellulose, and alginic acid. Again, the pH adjuster results in an initial pH outside the range of typical pH for urine whereby a color change can occur in the presence of urine.

In addition, the color-changing composition of the present disclosure includes a wettability agent. It has been surprisingly discovered that a composition of cellulose fibers treated with highly positively charged polymers and immobilized with pH indicators and pH adjusters does not have very good urine wettability, when considering the highly charged nature of the fibers. The materials of the present disclosure include a wettability agent that provides improved urine wettability. The wettability agent can be present in an amount of less than about 0.5 weight percent of the total weight of the treated fibers of the substrate, more particularly less than about 0.1 weight percent of the total weight of the treated fibers of the substrate, still more particularly less than about 0.01 weight percent of the total weight of the treated fibers of the substrate. For example, the color-changing composition can include a treatment composition that contains a water-soluble organic polymer (e.g., polysaccharides and derivatives thereof). The color-changing composition can also employ surfactants to enhance the hydrophilic nature of the fibers. Ionic surfactants (i.e., anionic, cationic, or amphoteric surfactants) and/or nonionic surfactants may be employed in the color-changing composition. Particularly suitable surfactants are nonionic surfactants, such as alkyl glycosides, ethoxylated alkylphenols, ethoxylated and propoxylated fatty alcohols, ethylene oxide-propylene oxide block copolymers, ethoxylated esters of fatty (C₈-C₁₈) acids, condensation products of ethylene oxide with long chain amines or amides, condensation products of ethylene oxide with alcohols, and mixtures thereof. Various specific examples of suitable nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and mixtures thereof.

In certain embodiments, alkyl glycosides are employed in the color-changing composition. Alkyl glycosides are generally prepared by reacting a monosaccharide, or a compound hydrolyzable to a monosaccharide, with an alcohol such as a fatty alcohol in an acid medium. For example, U.S. Pat. Nos. 5,527,892 and 5,770,543, which are incorporated herein in their entirety by reference thereto for all purposes, describe alkyl glycosides and/or methods for their preparation. Commercially available examples of suitable alkyl glycosides include Glucopon™ 220, 225, 425, 600 and 625, all of which are available from Cognis Corp. of Cincinnati, Ohio. These products are mixtures of alkyl mono- and oligoglucopyranosides with alkyl groups based on fatty alcohols derived from coconut and/or palm kernel oil. Glucopon™ 220, 225 and 425 are examples of particularly suitable alkyl polyglycosides. Glucopon™ 220 is an alkyl polyglycoside that contains an average of 1.4 glucosyl residues per molecule and a mixture of 8 and 10 carbon alkyl groups (average carbons per alkyl chain-9.1). Glucopon™ 225 is a related alkyl polyglycoside with linear alkyl groups having 8 or 10 carbon atoms (average alkyl chain-9.1 carbon atoms) in the alkyl chain. Glucopon™ 425 includes a mixture of alkyl polyglycosides that individually include an alkyl group with 8, 10, 12, 14 or 16 carbon atoms (average alkyl chain-10.3 carbon atoms). Glucopon™ 600 includes a mixture of alkyl polyglycosides that individually include an alkyl group with 12, 14 or 16 carbon atoms (average alkyl chain 12.8 carbon atoms). Glucopon™ 625 includes a mixture of alkyl polyglycosides that individually include an alkyl group having 12, 14 or 18 carbon atoms (average alkyl chain 12.8 carbon atoms). Still other suitable alkyl glycosides are available from Dow Chemical Co. of Midland, Mich. under the Triton™ designation or under the Tween™ designation.

One particular embodiment of a method for detecting the presence of urine will now be described in more detail. Initially, a urine test sample is applied to the substrate. As the urine flows through the substrate, the change in hydrogen ion concentration is detected by a pH indicator of the color-changing composition. Thus, the color change or color intensity change can be determined, either visually or with instrumentation to detect the presence of urine.

In certain embodiments, the substrate is configured to change color in less than about 5 minutes when contacted with urine, more particularly, in less than about 3 minutes when contacted with urine, still more particularly, in less than about 1 minute when contacted with urine.

The present disclosure provides simple, compact and cost-efficient materials for accurately detecting urine. The test result can be visible so that it is readily observed by the person performing the test in a prompt manner and under test conditions conducive to highly reliable and consistent test results.

In accordance with the present disclosure, one or more materials described herein can also be integrated into an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underzones, bedzones, bandages, absorbent drapes, and medical wipes; food service wipers; clothing articles; and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, etc.), and an absorbent core.

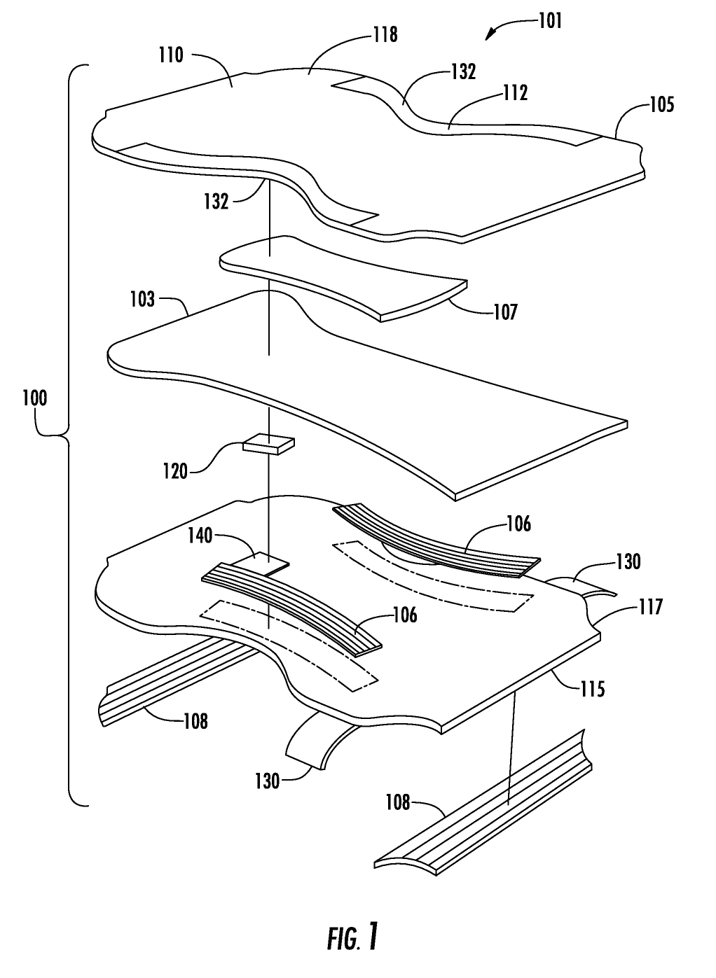

Various embodiments of an absorbent article that can be formed according to the present disclosure will now be described in more detail. For purposes of illustration only, an absorbent article is shown in FIG. 1 as a diaper 101. In the illustrated embodiment, the diaper 101 is shown as having an hourglass shape in an unfastened configuration. However, other shapes can of course be utilized, such as a generally rectangular shape, T-shape, or I-shape. As shown, the diaper 101 includes a chassis formed by various components, including an outer cover 117, bodyside liner 105, absorbent core 103, and surge layer 107. It should be understood, however, that other layers can also be used in exemplary embodiments of the present disclosure. Likewise, one or more of the layers referred to in FIG. 1 can also be eliminated in certain exemplary embodiments of the present disclosure.

The bodyside liner 105 is generally employed to help isolate the wearer's skin from liquids held in the absorbent core 103. For example, the liner 105 presents a bodyfacing surface that is typically compliant, soft feeling, and non-irritating to the wearer's skin. Typically, the liner 105 is also less hydrophilic than the absorbent core 103 so that its surface remains relatively dry to the wearer. As indicated above, the liner 105 can be liquid-permeable to permit liquid to readily penetrate through its thickness. Exemplary liner constructions that contain a nonwoven web are described in U.S. Pat. No. 5,192,606 to Proxmire, et al.; U.S. Pat. No. 5,702,377 to Collier, IV, et al.; U.S. Pat. No. 5,931,823 to Stokes, et al.; U.S. Pat. No. 6,060,638 to Paul, et al., and U.S. Pat. No. 6,150,002 to Varona, as well as U.S. Patent Application Publication Nos. 2004/0102750 to Jameson; 2005/0054255 to Morman, et al.; and 2005/0059941 to Baldwin, et al., all of which are incorporated herein in their entirety by reference thereto for all purposes.

The diaper 101 can also include a surge layer 107 that helps to decelerate and diffuse surges or gushes of liquid that can be rapidly introduced into the absorbent core 103. Desirably, the surge layer 107 rapidly accepts and temporarily holds the liquid prior to releasing it into the storage or retention portions of the absorbent core 103. In the illustrated embodiment, for example, the surge layer 107 is interposed between an inwardly facing surface 116 of the bodyside liner 105 and the absorbent core 103. Alternatively, the surge layer 107 can be located on an outwardly facing surface 118 of the bodyside liner 105. The surge layer 107 is typically constructed from highly liquid-permeable materials. Examples of suitable surge layers are described in U.S. Pat. No. 5,486,166 to Ellis, et al. and U.S. Pat. No. 5,490,846 to Ellis, et al., which are incorporated herein in their entirety by reference thereto for all purposes.

The outer cover 117 is typically formed from a material that is substantially impermeable to liquids. For example, the outer cover 117 can be formed from a thin plastic film or other flexible liquid-impermeable material. In one embodiment, the outer cover 117 is formed from a polyethylene film having a thickness of from about 0.01 millimeter to about 0.05 millimeter. The film can be impermeable to liquids, but permeable to gases and water vapor (i.e., “breathable”). This permits vapors to escape from the absorbent core 103, but still prevents liquid exudates from passing through the outer cover 117. If a more cloth-like feeling is desired, the outer cover 117 can be formed from a polyolefin film laminated to a nonwoven web. For example, a stretch-thinned polypropylene film can be thermally laminated to a spunbond web of polypropylene fibers.

Besides the above-mentioned components, the diaper 101 can also contain various other components as is known in the art. For example, the diaper 101 can also contain a substantially hydrophilic tissue wrapsheet (not illustrated) that helps maintain the integrity of the fibrous structure of the absorbent core 103. The tissue wrapsheet is typically placed about the absorbent core 103 over at least the two major facing surfaces thereof, and composed of an absorbent cellulosic material, such as creped wadding or a high wet-strength tissue. The tissue wrapsheet can be configured to provide a wicking layer that helps to rapidly distribute liquid over the mass of absorbent fibers of the absorbent core 103. The wrapsheet material on one side of the absorbent fibrous mass can be bonded to the wrapsheet located on the opposite side of the fibrous mass to effectively entrap the absorbent core 103. Furthermore, the diaper 101 can also include a ventilation layer (not shown) that is positioned between the absorbent core 103 and the outer cover 117. When utilized, the ventilation layer can help insulate the outer cover 117 from the absorbent core 103, thereby reducing dampness in the outer cover 117. Examples of such ventilation layers can include a nonwoven web laminated to a breathable film, such as described in U.S. Pat. No. 6,663,611 to Blaney, et al., which is incorporated herein in its entirety by reference thereto for all purposes.

In some embodiments, the diaper 101 can also include a pair of side panels (or ears) (not shown) that extend from the side edges 132 of the diaper 101 into one of the waist regions. The side panels can be integrally formed with a selected diaper component. For example, the side panels can be integrally formed with the outer cover 117 or from the material employed to provide the top surface. In alternative configurations, the side panels can be provided by members connected and assembled to the outer cover 117, the top surface, between the outer cover 117 and top surface, or in various other configurations. If desired, the side panels can be elasticized or otherwise rendered elastomeric by use of the elastic nonwoven composite of the present disclosure. Examples of absorbent articles that include elasticized side panels and selectively configured fastener tabs are described in PCT Patent Application WO 95/16425 to Roessler; U.S. Pat. No. 5,399,219 to Roessler et al.; U.S. Pat. No. 5,540,796 to Fries; and U.S. Pat. No. 5,595,618 to Fries, each of which is incorporated herein in its entirety by reference thereto for all purposes.

As representatively illustrated in FIG. 1, the diaper 101 can also include a pair of containment flaps 112 that are configured to provide a barrier and to contain the lateral flow of body exudates. The containment flaps 112 can be located along the laterally opposed side edges 132 of the bodyside liner 105 adjacent the side edges of the absorbent core 103. The containment flaps 112 can extend longitudinally along the entire length of the absorbent core 103, or can only extend partially along the length of the absorbent core 103. When the containment flaps 112 are shorter in length than the absorbent core 103, they can be selectively positioned anywhere along the side edges 132 of diaper 101 in a crotch region 110. In one embodiment, the containment flaps 112 extend along the entire length of the absorbent core 103 to better contain the body exudates. Such containment flaps 112 are generally well known to those skilled in the art. For example, suitable constructions and arrangements for the containment flaps 112 are described in U.S. Pat. No. 4,704,116 to Enloe, which is incorporated herein in its entirety by reference thereto for all purposes.

To provide improved fit and to help reduce leakage of body exudates, the diaper 101 can be elasticized with suitable elastic members, as further explained below. For example, as representatively illustrated in FIG. 1, the diaper 101 can include leg elastics 106 constructed to operably tension the side margins of the diaper 101 to provide elasticized leg bands which can closely fit around the legs of the wearer to reduce leakage and provide improved comfort and appearance. Waist elastics 108 can also be employed to elasticize the end margins of the diaper 101 to provide elasticized waistbands. The waist elastics 108 are configured to provide a resilient, comfortably close fit around the waist of the wearer.

The diaper 101 can also include one or more fasteners 130. For example, two flexible fasteners 130 are illustrated in FIG. 1 on opposite side edges of waist regions to create a waist opening and a pair of leg openings about the wearer. The shape of the fasteners 130 can generally vary, but can include, for instance, generally rectangular shapes, square shapes, circular shapes, triangular shapes, oval shapes, linear shapes, and so forth. The fasteners can include, for instance, a hook-and-loop material, buttons, pins, snaps, adhesive tape fasteners, cohesives, fabric-and-loop fasteners, etc. In one particular embodiment, each fastener 130 includes a separate piece of hook material affixed to the inside surface of a flexible backing.

The various regions and/or components of the diaper 101 can be assembled together using any known attachment mechanism, such as adhesive, ultrasonic, thermal bonds, etc. Suitable adhesives can include, for instance, hot melt adhesives, pressure-sensitive adhesives, and so forth. When utilized, the adhesive can be applied as a uniform layer, a patterned layer, a sprayed pattern, or any of separate lines, swirls or dots. In the illustrated embodiment, for example, the outer cover 117 and bodyside liner 105 are assembled to each other and to the absorbent core 103 using an adhesive. Alternatively, the absorbent core 103 can be connected to the outer cover 117 using conventional fasteners, such as buttons, hook and loop type fasteners, adhesive tape fasteners, and so forth. Similarly, other diaper components, such as the leg elastic members 106, waist elastic members 108 and fasteners 130, can also be assembled into the diaper 101 using any attachment mechanism.

Generally speaking, the materials of the present disclosure can be incorporated into the absorbent article in a variety of different orientations and configurations, so long as they are capable of receiving urine and providing a color change to a user or caregiver of the detection of urine. The visibility of the materials can be accomplished in a variety of ways. For example, in some embodiments, the absorbent article can include a transparent or translucent portion 140 (e.g., window, film, etc.) that allows the material to be readily viewed without removal of the absorbent article from the wearer and/or without disassembly of the absorbent article. In other embodiments, the material can extend through a hole or aperture in the absorbent article for observation. In still other embodiments, the material can simply be positioned on a surface of the absorbent article for observation.

Regardless of the particular manner in which it is integrated, urine can be directly discharged to a portion of the material 120, or can be discharged onto a component of the absorbent article into which the material 120 has been integrated. After a sufficient reaction time, the color can be detected to determine the presence of urine.

The present disclosure can be better understood with reference to the following examples.

### EXAMPLES

### Example 1

1 g dispersed cellulose fiber was combined with 0.5 ml of 20% Kymene in 60 ml water. The mixture was heated at 70° C. for two hours and filtered and washed three times with water. The washed fiber was re-suspended in an aqueous solution containing 0.4 mg/ml of bromocresol green and 4 mg/ml of citric acid for one hour. The fiber was then equally divided into three tubes, designated as tube 1, 2 and 3, respectively. 0.5 mg/ml of Tween-20 was added to tube 2. 200 mg/ml of Tween-20 was added to tube 3. The fiber in the three tubes was filtered. The three fiber sheets were then dried at 70° C. for one hour. One small piece of each sheet was cut and laid on a sheet of tissue and 200 μl of synthetic urine was applied to each piece, designated as sample 1, 2, and 3, respectively. It was found that the synthetic urine penetrated into the matrix of samples 2 and 3 much faster than sample 1, indicating better wettability with Tween-20 treatment. The color change from yellow/green to blue occurred faster for samples 2 and 3 than sample 1. However, more dyes were found to leach out of the fiber sheet into the tissue for sample 3 than for samples 1 and 2. Generally, the wettability enhancers should not exceed 0.5% of the total weight of the treated fiber. Therefore, the benefit of better wettability and negative effect of increased leaching needs to be carefully balanced using an appropriate amount of wettability enhancer.

### Example 2

0.5 ml, 0.1 ml and 0.02 ml of 20% Kymene was added to each of three tubes (designated as tube 1, 2, and 3, respectively) containing 600 mg dispersed cellulose fiber in 40 ml water. The samples were bath-sonicated at 50° C. for 3 hours, then filtered, and washed once by water. The filtered fiber was then dispersed in 40 ml water. 0.5 ml of bromocresol green (10 mg/ml) was added to each sample and incubated at room temperature for one hour. The fiber was filtered and washed once by water. The filtrant of sample 1 had almost no color, indicating all of the dye has been absorbed by the fiber. Some blue color was observed for the filtrant of sample 2. Strong blue color was observed for the filtrant of sample 3, suggesting a large amount of dye was not absorbed by the fiber. The fiber of samples 1 and 2 were deep blue while the fiber of the sample 3 was light blue. The filtered fiber of each sample was re-suspended into 18 ml water and 2 ml of 40 mg/ml of citric acid for half an hour. The fiber of sample 1 became light blue while the fibers of samples 2 and 3 became yellowish. A portion of the fibers were filtered and dried at 37° C. for an hour. The fiber sheets of samples 2 and 3 were yellow while the fiber sheet of sample 1 was yellowish blue. A piece of each fiber sheet was applied with 100 μl of hepes buffer (0.1M, pH: 7.2) and the color of all the pieces became deep blue. However, it was observed that it took a few minutes for the buffer to penetrate the whole fiber sheets, suggesting less desirable water wettability.

### Example 3

A portion of sample 2 (20 ml) from example 2 was added with 2% Triton-X-100 and incubated for 30 minutes, and then filtered and dried at 30° C. for an hour. A piece of the fiber sheet along with a piece of the fiber of sample 2 prepared in Example 2 was applied with 100 ml of hepes buffer. The buffer was found to instantaneously penetrate the fiber sheet with the Triton-X-100 treatment, while it took a few minutes for the buffer to completely penetrate the fiber sheet without the Triton-X-100 treatment.

### Example 4

A rectangular hole around the center of the outer cover of a Huggies® diaper was made by removing a portion of the outer cover. The fiber sheets prepared in above examples were then inserted against the absorbent core. A transparent Scotch tape was used to cover and secure the materials against the absorbent core. 10 ml of urine was applied from the center of the inner side. The color of a large portion (>50%) of the fiber pieces made as sample 2 with Tween-200 treatment changed color from yellow to blue in fewer than 5 minutes. Almost the whole piece changed color to blue from yellow 5 minutes after additional 10 ml of synthetic urine. Only a very small spotted portion (<10%) of the fiber pieces made as sample 2 without Tween-200 treatment changed color from yellow to blue in more than 5 minutes. Less than 30% of the fiber pieces made as sample 2 without Tween-200 treatment changed color from yellow to blue 30 minutes after an additional 10 ml of synthetic urine was added.

### Example 5

Each of three tubes (designated as tube 1, 2, and 3) containing 600 mg dispersed fiber in 40 ml water was added with 200 μl, 100 μl and 50 μl of 20% Kymene. The mixtures were heated at 80° C. for 30 minutes. Each sample was divided equally into two 20 ml samples. One of each was filtered and washed once with water. Each of the six samples was added with 1.5 ml of a mixture containing 2 ml bromocresol green (10 mg/ml), 8 ml of citric acid (40 mg/ml) and 2 ml Triton-X-100 (2%). The samples were allowed to incubate at room temperature for 20 minutes. The samples were then filtered and dried at 37° C. for two hours. The dried fiber sheets made from tubes 1, 2 and 3 with the excess Kymene filtered showed strong bluish yellow, bluish-yellow and yellow, respectively. The color of a piece of the fiber sheet made of those samples changed color to deep blue upon addition of 200 μl synthetic urine. All of the dried fiber sheets made from the tube 1, 2 and 3 without removing the excess Kymene showed very light white/blue color. Very little dye was absorbed by the fibers. The color of a piece of the fiber sheet made of those samples did not change color upon addition of 200 μl synthetic urine.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure so further described in such appended claims.

What is claimed is:

1. A method for detecting urine, the method comprising:
contacting urine with a substrate, the substrate comprising charged cellulosic fibers having a color-changing composition immobilized thereon, the color-changing composition comprising a pH indicator, a pH adjuster, and a wettability agent, wherein the pH indicator is configured to change color when contacted with urine;
determining the presence of urine based on a change in color of the pH indicator.

2. The method of claim 1, wherein the pH indicator comprises bromochlorophenol blue, bromophenol blue, methyl orange, methyl yellow, alizarine yellow GG, bromocresol green, phloxine B, congo red, ethyl orange, nile blue A, aniline blue W.S., or combinations thereof.

3. The method of claim 1, wherein the cellulosic fibers comprise eucalyptus pulp fibers.

4. The method of claim 1, wherein the cellulosic fibers are charged with a positively charged polyamide epicholorohydrin polymer.

5. The method of claim 1, wherein the pH adjuster comprises an inorganic acid, an organic acid, a polymeric acid, or combinations thereof.

6. The method of claim 1, wherein the pH adjuster comprises an organic acid comprising oxalic acid, citric acid, salicylic acid, tartaric acid, or combinations thereof.

7. The method of claim 1, wherein the pH adjuster comprises an inorganic base, an organic base, or combinations thereof.

8. The method of claim 1, wherein the wettability agent comprises a non-ionic surfactant.

9. The method of claim 1, wherein the wettability agent is present in an amount of less than about 0.5 weight percent of the substrate.

10. The method of claim 1, wherein the wettability agent is present in an amount of less than about 0.1 weight percent of the substrate.

11. The method of claim 1, wherein the wettability agent is present in an amount of less than about 0.01 weight percent of the substrate.

12. The method of claim 1, wherein the substrate is configured to change color in less than about 5 minutes when contacted with urine.

13. The method of claim 1, wherein the substrate is configured to change color in less than about 1 minute when contacted with urine.

14. An absorbent article capable of determining the presence of urine comprising:
a substantially liquid impermeable layer;
a liquid permeable layer;
an absorbent core positioned between the substantially liquid impermeable layer and the liquid permeable layer; and
charged cellulosic fibers integrated into the article and positioned such that the charged cellulosic fibers are in fluid communication with the urine when provided by a wearer of the article, the charged cellulosic fibers having a color-changing composition immobilized thereon, the color-changing composition comprising a pH indicator, a pH adjuster, and a wettability agent, wherein the pH indicator is configured to change color when contacted with urine.

15. The absorbent article of claim 14, the pH indicator comprises bromochlorophenol blue, bromophenol blue, methyl orange, methyl yellow, alizarine yellow GG, bromocresol green, phloxine B, congo red, ethyl orange, nile blue A, aniline blue W.S., or combinations thereof.

16. The absorbent article of claim 14, wherein the cellulosic fibers comprise eucalyptus pulp fibers.

17. The absorbent article of claim 14, wherein the cellulosic fibers are charged with a positively charged polyamide epicholorohydrin polymer.

18. The absorbent article of claim 14, wherein the pH adjuster comprises an inorganic acid, an organic acid, a polymeric acid, or combinations thereof.

19. The absorbent article of claim 14, wherein the pH adjuster comprises an inorganic base, an organic base, or combinations thereof.

20. The absorbent article of claim 14, wherein the wettability agent comprises a non-ionic surfactant.

21. The absorbent article of claim 14, wherein the wettability agent is present in an amount of less than about 0.5 weight percent of the charged cellulosic fibers.

22. The absorbent article of claim 14, wherein the absorbent article defines a window through which at least a portion of the charged cellulosic fibers are observable.
